(19) United States
(12) Patent Application Publication
(10) Pub. No.: US 2025/0248595 A1
(43) Pub. Date: Aug. 7, 2025

(54) LIGHT SECURE EYE TRACKER

(71) Applicant: Vision Products, LLC, Campbell, CA (US)

(72) Inventors: Michael P. Browne, San Mateo, CA (US); Kevin Lee Mellott, San Jose, CA (US)

(21) Appl. No.: 18/432,758

(22) Filed: Feb. 5, 2024

(51) Int. Cl. A61B 3/113; G02B 26/04; G02B 27/01

(52) U.S. Cl. CPC A61B 3/113; G02B 26/04; G02B 27/0101; G02B 2027/0118

(57) ABSTRACT

An eye tracking system may include a light source with adjustable emission and positioned to illuminate an eye of a user with light. The system may include a light detector positioned to generate data corresponding to the eye of the user. The system may include a shutter with adjustable transmission and positioned between the eye of the user and an external environment. The system may include a control module configured to control the light source and the shutter to synchronize repeated variations of transmission of the shutter with repeated variations of emission from the light source.

### BACKGROUND

### 1. Technical Field

This disclosure relates generally to eye tracking systems and extended reality systems.

### 2. Description of Related Art

Advances in display technologies and mobile computing systems have facilitated the development of augmented reality (AR) systems that can present a user with a view of the physical environment augmented with computer-generated information such as text, data, graphics, images, video, etc. Examples of such information include, maps, GPS data, and photos. This supplemental content may be presented in a manner wherein the user perceives the virtual objects to be superimposed on or adjacent to the view of the real-world objects in front or surrounding the user. Presentation of this additional content can be provided in real-time.

### BRIEF DESCRIPTION OF THE DRAWINGS

Embodiments of the disclosure have other advantages and features which will be more readily apparent from the following detailed description and the appended claims, when taken in conjunction with the examples in the accompanying drawings, in which:

FIG. 1 is a conceptual block diagram of a light secure eye tracking system, according to some embodiments.

FIG. 2A is a conceptual block diagram of the light secure eye tracking system where the shutter is in a transmissive state, the display is in a dark state, and an electromagnetic (EM) source is in a dark state, according to some embodiments.

FIG. 2B is a conceptual block diagram of the light secure eye tracking system where the shutter is in a blocking state, the display is in a display state, and an EM source is in a light state, according to some embodiments.

FIG. 3 illustrates a method for implementing a light secure eye tracking system, according to one or more embodiments.

FIG. 4 is a block diagram illustrating an embodiment of components of an example machine.

### DETAILED DESCRIPTION

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

### Example Eye Tracking Systems

Embodiments herein relate to “light secure” eye tracking systems (e.g., mounted to a user's head) that block or reduce light emission into the external environment while still allowing the user to operate in the environment. More specifically, light used to illuminate the user's eye (e.g., infrared light) for eye tracking purposes is prevented or reduced from leaving the system and entering the external environment. Some embodiments further relate to light secure eye tracking systems with extended reality capabilities (e.g., augmented reality, mixed reality, or virtual reality). In addition to preventing or reducing light leakage from the eye tracking components, these embodiments may prevent light from the extend reality components (e.g., virtual objects displayed to the user) from leaking into the external environment.

Among other advantages, these systems increase user privacy by reducing or preventing the ability for people or sensors in the external environment to detect eye tracking information of the user or extended reality images displayed to the user. In addition to helping protect a user's privacy, these systems may help a user avoid detection by people or sensors in the external environment. For example, if the user (e.g., a soldier) is operating in the external environment at night, preventing light leakage may inhibit detection (e.g., by night vision goggles), while still providing the user with the advantages of a system with eye tracking and extended reality capabilities. Examples of light secure systems are further described below.

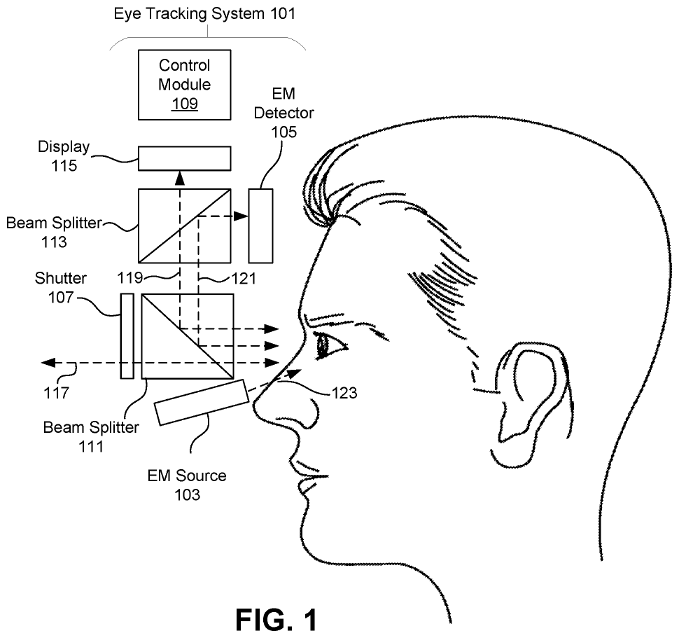

FIG. 1 is a conceptual block diagram of a light secure eye tracking system 101, according to some embodiments. In the example of FIG. 1, the eye tracking system 101 includes an electromagnetic (EM) source 103, an EM detector 105, a shutter 107, a control module 109, a beam splitter 111, a beam splitter 113, and a display 115 (although the example eye tracking system 101 of FIG. 1 includes one of each of these components, an eye tracking system (e.g., 101) can include two or more of any of these components). Note that the components in FIG. 1 are not drawn to scale relative to each other and relative to the user.

The eye tracking system 101 may include additional, fewer, or different components than illustrated in FIG. 1. For example, although FIG. 1 includes two beam splitters to direct light, different optical arrangements may be used. Additionally, one or more of the components may be positioned differently than as illustrated in FIG. 1. For example, the positions of the EM detector 105 and the source 103 may be switched, or the positions of the display 115 and the EM detector 105 may be switched. In another example, the display 115 is below or to the side of the user's eye. In another example, the EM detector 105 is positioned differently so it is directed toward the user's eye (e.g., in a similar position as the EM source 103 (e.g., the EM detector is adjacent to or integrated into the EM source 103, the display 115, or some combination thereof)). In this example, beam splitter 113 may be removed from the eye tracking system 101. In another example of components being positioned differently than as illustrated in FIG. 1 (e.g., in addition to, or alternative to, the previous examples), the EM source 103 is adjacent to or integrated into the EM detector 105, the display 115, or some combination thereof). In some embodiments, the components are positioned to increase the user's view of the external environment.

The components of the eye tracking system 101 may be supported by a frame (not illustrated) that can be worn on or around the user's head (e.g., the eye tracking system 101 is a head-mounted device (HMD)). Furthermore, while the components are illustrated as blocks, the components can take on many different shapes and configurations. For example, the eye tracking system 101 may be similar to a pair of glasses or goggles.

The eye tracking system 101 includes multiple optical paths, indicated by the dashed arrows in FIG. 1. A first optical path 117 is between the external environment and the user's eye. This optical path 117 enables light from the external environment to propagate through the shutter 107 and through the beam splitter 111 to reach the user's eye. The second optical path 119 is between the display 115 and the user's eye. This optical path 119 enables light from the display to 115 propagate downward through the beam splitter 113 and be directed horizontally by beam splitter 111 toward the user's eye. The third optical path 121 is between the EM detector 105 and the user's eye. This optical path 121 enables light from the user's eye (e.g., EM source light reflected off the eye) to be directed upward by beam splitter 111 and directed toward the EM detector 105 by the beam splitter 113. The fourth optical path 123 is between the EM source 103 and the user's eye, thus enabling light from the EM source 103 to be directed toward the user's eye.

The EM source 103 is a device that emits electromagnetic waves. The EM source 103 may also be referred to as a “light source” since the term “light,” as used herein, includes electromagnetic wavelengths outside of the visible spectrum (e.g., infrared light). The EM source 103 may emit electromagnetic waves in a limited range of wavelengths (e.g., visible wavelengths, infrared wavelengths, ultraviolet wavelengths, or some combination thereof). The EM source 103 is positioned in the eye tracking system 101 to illuminate the user's eye (e.g., with eye-safe wavelengths, with an amount of light that is eye-safe, or some combination thereof), thus enabling the EM detector 105 to generate data corresponding to the user's illuminated eye (e.g., to capture illuminated images of the user's eye). To avoid distracting the user, the EM source 103 emits light outside of the visible spectrum in some embodiments. For example, the EM source 103 (e.g., only) emits infrared wavelengths. In some embodiments, the EM source 103 emits infrared wavelengths longer than 950 nanometers (nm) to reduce the likelihood that EM source wavelengths which unintentionally leak into the environment are detected by a night vision system (or other near infrared detection systems) since most night vision systems do not detect wavelengths longer than 950 nm.

The EM source 103 may be configured to emit light periodically or to adjust its emission over certain time periods (e.g., it is configured to emit pulses as specific times). More specifically, the EM source 103 may have a light state, during which is emits light (e.g., a pulse), and a dark state, during which it emits a reduced amount of light or no amount of light compared to the light state (e.g., less than 1% or 5% of the average light emitted during the light state). Emission of light by the EM source 103 may be synchronized with other components of the eye tracking system 101 as further described below. The EM source 103 may be coupled to and controlled by the control module 109. For example, the EM source 103 is controlled by the control module 109 to adjust its emissions and to be synchronized with the shutter 107.

The EM detector 105 (also referred to as an EM sensor 105) is positioned in the eye tracking system 101 to capture light from the user's eye (e.g., light from EM source 103 reflected off the user's eye) and generate data corresponding to said captured light (referred to as “eye data”). The EM detector 105 may also be referred to as a “light detector” since the term “light,” as used herein, includes electromagnetic wavelengths outside of the visible spectrum (e.g., infrared light). If the EM source 103 emits light in a specific wavelength range (e.g., infrared wavelengths), the EM detector 105 may be specialized to capture light in that wavelength range (e.g., the EM detector 105 is an infrared camera). The EM detector 105 is configured to generate eye data (e.g., images) when the EM source 103 emits lights. The EM detector 105 may be coupled to and controlled by the control module 109. For example, the EM detector 105 is controlled by the control module 109 to generate data when the EM source 103 emits light. The EM detector 105 may be one or more cameras, one or more imaging sensors (e.g., that captures images of the user's eye), one or more edge detectors, one or more event sensors, one or more photodiodes, or some combination thereof.

The display 115 can display images that are directed to the user. The display 115 may have a display state, during which it displays images, and a dark state, during which it emits a reduced amount of light or no amount of light compared to the display state (e.g., less than 1% or 5% of the average light emitted during the display state). The display and dark states may be controlled by the control module 109. The display 115 may be, for example, a liquid crystal display (LCD), an organic light emitting diode (OLED) display, or a micro-LED device. The eye tracking system 101 may have any number of displays 115. The images may be extended reality images (e.g., mixed reality, augmented reality, or virtual reality images). For example, the images displayed by the display 115 include information, such as text, images, graphics, and videos, that modify or augment the user's view of the external environment. The information may be specific to the user's view of the external environment, such as information that identifies people and objects, and it may assist the user to navigate or operate in the external environment. The images may be generated by the control module 109 or another computer system (not illustrated) and transmitted to the display 115.

In some embodiments, the EM source 103 and the display 115 are combined into a single component. In these embodiments, light from the display 115 may be used to illuminate the user's eye, thus enabling the EM detector 105 to generate data corresponding to the user's illuminated eye (e.g., to capture illuminated images of the user's eye). For these embodiments, any descriptions herein related to synchronization of the EM source 103 with the shutter 107 may be applicable to the single combined component (e.g., the display 115). Light to illuminate the eye from the single combined component may come from one or more display pixels of the display 115 (e.g., pixels also used to generate images for the display 115). Additionally, or alternatively, the single component may include one or more light sources configured to illuminate the eye (e.g., light sources not used to generate images for the display 115). These light sources may be, for example, between display pixels of the display 115 or along one or more edges of the display 115. If it is desirable for the eye to be illuminated with light outside of the visible spectrum (e.g., infrared light), the display 115 may include one or more light sources (e.g., pixels) configured to emit wavelengths outside of the visible spectrum (e.g., infrared light). Among other advantages, the EM source 103 and the display 115 integrated into a single component may reduce the number of components of the eye tracking system 101, thus making the system 101 less complex and smaller (a smaller system 101 may enable the user to have a greater view of the external environment). Furthermore, light to illuminate the eye will be emitted closer to the optical axis of the eye compared to the arrangement in FIG. 1 (where the EM source 103 is below the eye), which may reduce the number of light sources used to illuminate the eye or the total amount of light used to illuminate the eye.

In some embodiments, the EM detector 105 is adjacent to or integrated with the display 115 (e.g., the EM detector 105 is between display pixels of the display 115 or along one or more edges of the display 115)). For example, the EM detector 105 and the display 115 are positioned at the same image plane. Thus, in these embodiments, the EM detector 105 and the display 115 may have the same optical path (e.g., 119) or share at least a portion of their optical paths. Among other advantages, these embodiments may reduce the number of components, may make the system 101 less complex, may increase optical alignment of the system (e.g., aligning the combined component results in the EM detector 105 and the display 115 both being aligned with the eye), may make the system 101 smaller (e.g., the beam splitter 113 is removed and the EM detector 105 and display 115 may both be positioned above the beam splitter 111), or some combination thereof. Additionally, or alternatively, the EM detector 105, the EM source 103, and the display 115 may all be combined into a single component, thus enabling all three individual components to be on the same optical axis (e.g., 119) and further reducing the size and complexity of the system 101.

The beam splitter 111 combines images from the display 115 with light from the external environment (not necessarily at the same time) by directing light (via optical path 117) from the external environment to the user's eye and by directing light (via optical path 119) from the display 115 to the user's eye, thus enabling the user to view images from the display that appear to be in the external environment. The beam splitter 111 also directs light from the user's eye upward to be captured by the EM detector 105. The beam splitter 113 directs light from the user's eye (directed upward by the beam splitter 113) toward the EM detector 105. The beam splitter 113 also allows light from the display 115 to pass through it so that images can be directed to the user's eye (by the beam splitter 111).

One or both beam splitters 111, 113 may include a partially metallized mirror, a dielectric coating, a dichroic coating, an interference coating, or some combination thereof disposed on a transmissive material, a partially transmissive mirror, a waveguide device, a polarized beam combiner, or some combination thereof. Additionally, a beam splitter (e.g., 111 or 113) may include one or more focusing and/or collimating lenses. In some embodiments, a beam splitter has optical power that magnifies or shrinks images. The beam splitters 111, 113 may direct light to and from one or both eyes of the user.

The shutter 107 has adjustable light transmission and is positioned in the eye tracking system 101 along optical path 117. More specifically, the shutter 107 is disposed between the beam splitter 111 and the external environment, thus enabling light from the EM source 103 and the display 115 to propagate to the user's eye while also preventing or reducing light leakage into the external environment (e.g., preventing light from the display 115 or the EM source 103 reflected from the user's eye or facial features from entering the environment).

When the shutter 107 is in a transmissive state, the shutter 107 allows light to propagate from the external environment to the user's eye and vice versa. During a transmissive state, the shutter 107 may allow enough light for the user to view the external environment. When the shutter 107 is in a blocking state, the shutter 107 (at least partially) blocks light from propagating into the external environment (e.g., the shutter blocks more than 90%, 95%, or 99% of incident light from propagating through the shutter). The shutter 107 may be configured to transition between the transmissive state and the blocking state. Furthermore, the shutter 107 may be synchronized with the EM source 103, the display 115, the EM detector 105, or some combination thereof. The shutter 107 may be coupled to and controlled by the control module 109. For example, the shutter 107 is controlled by the control module 109 to transition states in synchronization with the EM source 103, the display 115, the EM detector 105, or some combination thereof.

The shutter may transition states at time intervals that are comparable to or less than the persistence of human vision, which is approximately 1/16 of a second. For example, the shutter 107 transitions from a first state (e.g., the transmissive state) to a second state (e.g., the blocking state) and back to the first state faster than the unaided human eye can detect. For example, the shutter 107 can be transitioned from a first state to a second state and back to the first state at a time intervals between about 1 microsecond and about 100 milliseconds (or any values in this range). Other components (e.g., 103 and 115) may also be capable of transitioning states at these time intervals.

In some embodiments, the transmissive state of the shutter 107 is not a state where the shutter is fully open (in other words, where the shutter 107 allows the maximum amount of light to propagate through). In these embodiments, the transmissive state may block a small portion of light (e.g., no more than 10% of incident light) from propagating through the shutter. Blocking a small portion of light may help control the amount of light seen by the user (e.g., to prevent images from the display from being washed out by external light).

The shutter 107 may be configured to block wavelengths of light emitted by the EM source 103 (e.g., infrared light) and by the display 115 (e.g., RGB wavelengths) when the shutter 107 is in the blocking state. In some cases, to block wavelengths from both components, the shutter 107 may include multiple shutters e.g., a first shutter configured to block wavelengths from the EM source 103 and a second shutter configured to block wavelengths from the display 115.

The shutter 107 can be any form of electro-optical shutter. For example, the shutter 107 is a liquid crystal shutter (e.g., it includes a twisted nematic (TN) liquid crystal element and/or a ferroelectric liquid crystal element). The shutter 107 may also include polarizers (e.g., to help block visible and infrared wavelengths). The shutter 107 may additionally include light absorbers to further decrease leakage of light reflected from the user.

Although not illustrated in FIG. 1, the eye tracking system 101 may include additional components to block or reduce light from propagating into the external environment, such as a housing, a frame, eyecups, baffles, or some combination thereof. For example, the eye tracking system 101 includes a component in front of beam splitter 113 to prevent display light directed forward by the beam splitter 113 from propagating into the environment. If either of the display 115 or the EM source 103 emit infrared wavelengths, those additional components may be configured to block infrared wavelengths as well. For example, an eyecup includes rubber or cloth material to block infrared wavelengths. In another example, a baffle or frame includes rubber, cloth, metal, plastic, or some combination thereof to block infrared wavelengths.

Additional details on example light secure systems can be found in U.S. Pat. No. 10,451,878, which is incorporated by reference herein in its entirety.

In some embodiments, the shutter 107 transforms the eye tracking system 101 from an optical see-through system (e.g., HMD) to an electronic see-through system (e.g., HMD) (also referred to as video see-through system). For example, the shutter 107 remains in a blocking state for a prolonged time period to occlude the user's view of the external environment. The eye tracking system 101 may then use outward facing cameras and the display 115 to display images of the external environment and/or electronic information to the user.

The eye tracking system 101 may include one or more optical filters. For example, a filter may be positioned along optical path 121 between the beam splitter 113 and the EM detector 105 and configured to block light emitted by the display 115 to prevent or reduce display light reflected off the user from being captured by the EM detector 105. This filter may result in the EM detector 105 mainly capturing EM source light reflected from the user (assuming the display 115 and the EM source 103 emit different wavelengths). In another example, a filter may be positioned adjacent to the shutter 107 and along optical path 117. The filter may be configured to block light wavelengths emitted by the EM source 103 (e.g., assuming the EM source 103 emits light outside of the visible spectrum), thus helping prevent or reduce light from the EM source 103 reflecting off the user and propagating into the external environment. This filter may work with in conjunction with the shutter 107. For example, if both are configured to block wavelengths from the EM source 103 then they may block more light together compared to just the shutter 107. In another example, if the shutter 107 is inefficient at blocking EM source light or not configured to block wavelengths from the EM source 103, the filter may be used to block EM source light while the shutter 107 is used to block display light.

The control module 109 is configured to control the display 115, the EM detector 105, the shutter 107, the EM source 103, or some combination thereof (as previously stated, the control module 109 may be coupled to any of these components as well). Among other control operations, the control module 109 may synchronize operations of any combination of components. Although the control module 109 is illustrated in the figures as a single component, the control module may be multiple components working in conjunction. Additionally, or alternatively, one or more functionalities of the control module 109 may be implemented by components of the eye tracking system 101, such as the EM source 103, the EM detector 105, the shutter 107, the display 115, or some combination thereof.

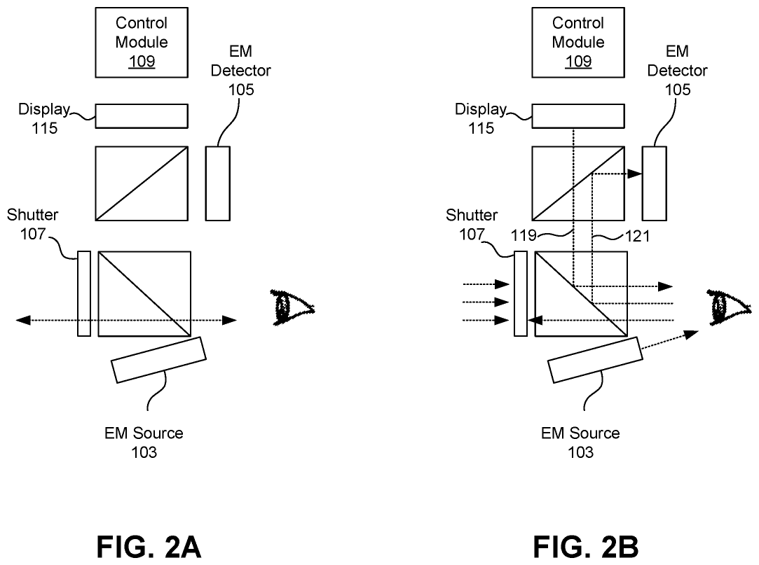

In some embodiments, the control module 109 synchronizes repeated variations of transmission of the shutter 107 (e.g., transitioning to different states) with repeated variations of emission from the EM source 103 (e.g., and the EM detector 105 generating data of the user's eye). For example, at a first time period, the shutter 107 is in a transmissive state and the EM source 103 is in a dark state. For example, see FIG. 2A, where the dotted lines represent light rays. At a subsequent second time period, the shutter 107 is in a blocking state, the EM source 103 emits a pulse of light (e.g., a light state), and the EM detector 105 generates data corresponding to the illuminated eye (e.g., see FIG. 2B, where the dotted lines represent light rays). At a subsequent third time period, the shutter 107 is back in a transmissive state and the EM source 103 is in a dark state. The control module 109 may control the shutter 107, EM source 103, and EM detector 105 accordingly such that the user does not notice the time periods or their transitions. The user may simply perceive seeing the external environment. Thus, the eye tracking system 101 can generate data corresponding to (e.g., illuminated images of) the user's eye without light leaking into the external environment, while still allowing the user to view the external environment (e.g., enough to move around and interact with objects in the environment).

In addition to being a light secure eye tracking system, the system 101 is also capable of being a light secure extended reality system. As previously mentioned, the display 115 and beam splitters 111, 113 may be used to display images to the user that appear to be overlaid onto or in the external environment. To do this, the control module 109 may synchronize repeated variations of the shutter 107 with repeated variations of the EM source 103, repeated variations of the display 115, and data generation by the EM detector 105. As previously mentioned, the transition rates and time periods for these variations may be faster than human perception (e.g., such that the user does not notice the time periods or their transitions). Thus, the user may perceive the external environment and virtual objects simultaneously (even though they may occur during different time periods to enable light security).

In a first example of a light secure extended reality system, the EM source 103 and the display 115 are in light states at substantially the same time while the shutter 107 is in a blocking state (e.g., see FIG. 2B) but are in dark states when the shutter 107 is in a transmissive state (e.g., see FIG. 2A). In a more specific example, when the shutter 107 is in a transmissive state, the EM source 103 may emit a reduced amount of light or no light (e.g., a dark state), and the display 115 may be in a dark state (that emits a reduced amount or no light). When the EM source 103 emits light (e.g., a light state), the EM detector 105 may generate data corresponding to the eye, the display 115 may be in the display state, and the shutter 107 may be in a blocking state that blocks stray or scattered light produced by the EM source 103 and/or the display 115 from leaking in the external environment. Additionally, or alternatively, the EM source 103 and the display 115 may emit light at different times when the shutter 107 is in a blocking state (but neither emit light when the shutter 107 is in a transmissive state) as the shutter 107 repeatedly transitions between states. This may avoid mixing of the display light with the EM source light. In a specific example, when the shutter 107 is in a transmissive state, the EM source 103 may emit a reduced amount of light or no light (e.g., a dark state), and the display 115 may be in the dark state. When the EM source 103 emits light (e.g., a light state), the EM detector 105 may generate data of the eye, the display 115 may be in the dark state, and the shutter 107 may be in a blocking state. When the display 115 is in the display state, the EM source 103 may emit a reduced amount of light or no light, and the shutter 107 may be in the blocking state. Additionally, or alternatively, when the shutter 107 is in a blocking state, during a first time period of the blocking state, the EM source 103 may emit light, the EM detector 105 may generate data of the eye, and the display 115 may be in the dark state. Furthermore, at a second time period of the blocking state (e.g., subsequent to or prior to the first time period), the EM source 103 may emit a reduced amount of light or no light and the display 115 may be in the display state.

Some of the previous examples assume that both the display 115 and the EM source 103 emit light when the shutter 107 is in a single blocking state. However, this is not required. If the shutter 107 is alternating between blocking and transmissive states, the display 115 and the EM source 103 may alternative emitting light during the blocking states. For example, at a first time period: the shutter 107 is in a blocking state, the EM source 103 is in a light state, the EM detector 105 generates data of the user's eye, and the display 115 is in the dark state (e.g., during the entirety of that blocking state). At a second time period subsequent to the first time period: the shutter 107 is in a transmissive state, the EM source 103 is in a dark state, and the display 115 is in the dark state. And at a third time period subsequent to the first time period and the second time period: the shutter 107 is in a blocking state, the EM source 103 is in a dark state (e.g., during the entirety of that blocking state), and the display 115 is in the display state.

The duty cycles of the shutter states may be configured so that the user perceives the external environment well enough to move and interact in the external environment (e.g., the user perceives an uninterrupted view of the external environment). For example, if the transmissive state durations are too short, a user may perceive flicker. Similarly, if the blocking state durations are too long, the external environment may appear unacceptably dimmed to the user.

The above examples describe light from the EM source 103 and the display 115 being prevented from leaking into the external environment (e.g., via synchronization with the shutter 107). However, this is not required. In some embodiments, the eye tracking system 101 is configured to block light only from the EM source 103 or light only from the display 115 (e.g., in situations where it is desirable to prevent infrared light from the EM source 103 leaking into the environment but acceptable for visible light from the display 115 to leak into the environment). For example, the shutter 107 is synchronized to block light from the EM source 103 but is not synchronized with the display 115.

To track the user's eye, the control module 109 may receive eye data from the EM detector 105 (e.g., an image captured by the EM detector 105) and analyze that image to determine a gaze of the user. Although embodiments herein are generally described in the context of eye tracking systems, this disclosure is not limited to eye tracking systems. For example, in addition to, or alternative to, eye tracking, the control module 109 may analyze the eye data to determine an identify of the user (e.g., analyzing the iris in a captured image).

### Example Methods

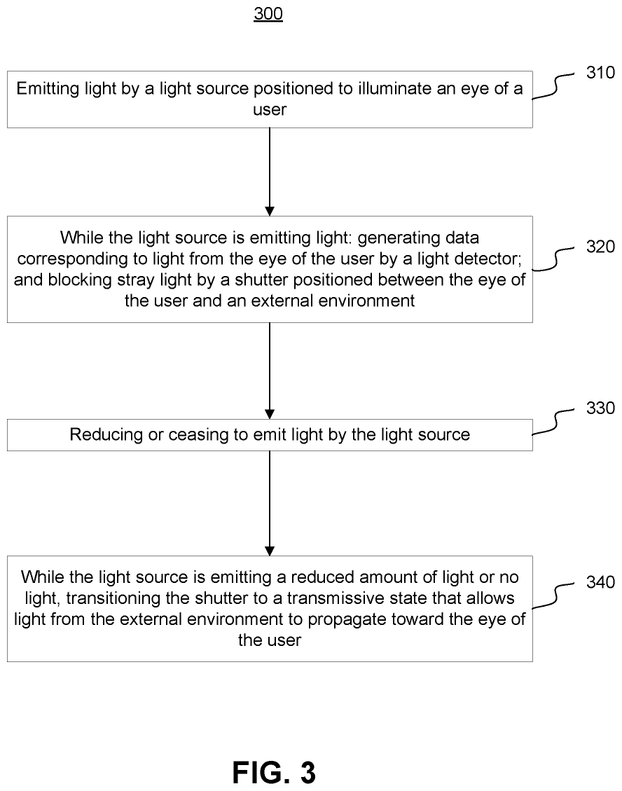

FIG. 3 illustrates a method 300 for implementing a light secure eye tracking system (e.g., 101), according to one or more embodiments. In the example of FIG. 3, the method 300 is performed by a control module (e.g., 109) controlling components. Instructions for the method 300 may be program code stored (e.g., on a non-transitory computer readable storage medium) that is executable by a computer system (e.g., control module 109). The steps may be performed in different orders, and the method 300 can include greater or fewer steps than described herein.

At step 310, a light source (e.g., 103) positioned to illuminate an eye of a user emits light.

At step 320, while the light source is emitting light, a light detector (e.g., 105) generates data corresponding to light from the eye of the user (e.g., captures one or more images of the eye of the user) and a shutter (e.g., 107) positioned between the eye of the user and an external environment blocks stray light propagating toward the external environment (e.g., reflected off the user's eye of facial features).

At step 330, the light source reduces or ceases to emit light.

At step 340, while the light source is emitting a reduced amount of light or no light, the shutter transitions to a transmissive state that allows light from the external environment to propagate toward the eye of the user.

Other aspects include components, devices, systems, improvements, methods, processes, applications, computer readable mediums, and other technologies related to any of the above.

### Computing System Architecture

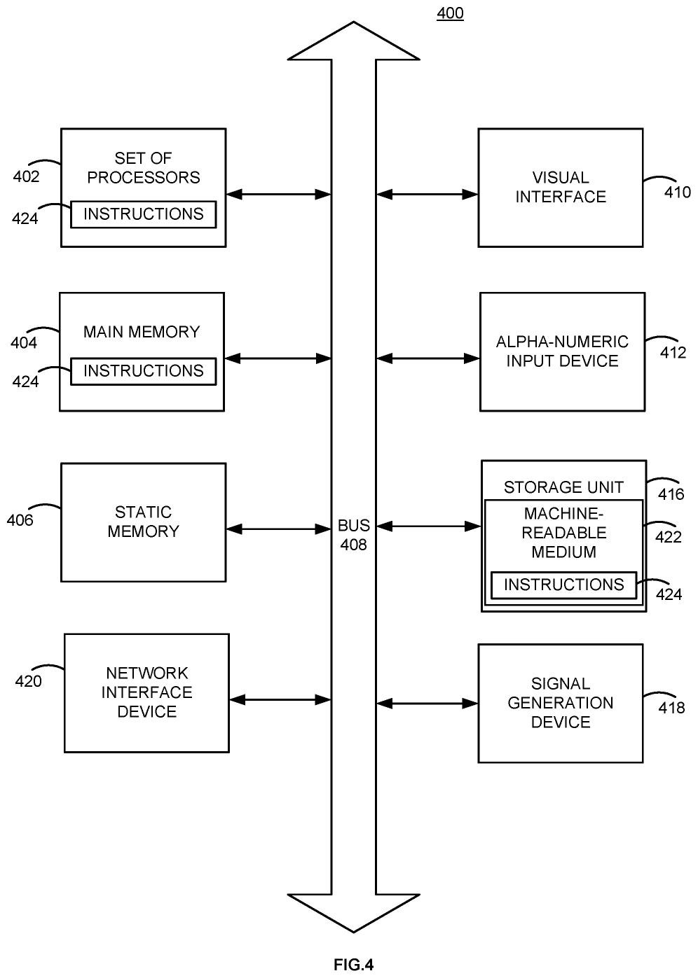

FIG. 4 is a block diagram illustrating an embodiment of components of an example machine able to read instructions from a machine-readable medium and execute them by a set of one or more processors (or controller). Specifically, FIG. 4 shows a diagrammatic representation of a machine in the example form of a computer system 400 (also “computing system 400”) within which program code (e.g., software) for causing the machine to perform any one or more of the methodologies discussed herein may be executed. The computer system 400 may be used for one or more components described herein, such as the control module 109, display 115, EM detector 105, shutter 107, and the EM source 103. The program code may be comprised of instructions 424 executable by a set of one or more processors 402 (e.g., which can execute the instructions individually or collectively) of the computer system 400. In alternative embodiments, the machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The machine may be a computing system capable of executing instructions 424 (sequential or otherwise) that specify actions to be taken by that machine. Further, while only a single machine is illustrated, the term “machine” shall also be taken to include any collection of machines that individually or jointly execute instructions 424 to perform any one or more of the methodologies discussed herein.

The example computer system 400 includes a set of one or more processors 402 (e.g., including one or more central processing units (CPUs), one or more graphics processing units (GPUs), one or more digital signal processors (DSPs), one or more application specific integrated circuits (ASICs), one or more radio-frequency integrated circuits (RFICs), one or more field programmable gate arrays (FPGAs), or some combination thereof), a main memory 404, and a static memory 406, which are configured to communicate with each other via a bus 408. The computer system 400 may further include visual display interface 410. The visual display interface may include a software driver that enables (or provide) user interfaces to render on a screen either directly or indirectly. The visual display interface 410 may interface with a touch enabled screen. The computer system 400 may also include input devices 412 (e.g., a keyboard a mouse), a storage unit 416, a signal generation device 418 (e.g., a microphone and/or speaker), and a network interface device 420, which also are configured to communicate via the bus 408.

The storage unit 416 includes a machine-readable medium 422 (e.g., magnetic disk or solid-state memory) on which is stored instructions 424 (e.g., software) embodying any one or more of the methodologies or functions described herein. The instructions 424 (e.g., software) may also reside, completely or at least partially, within the main memory 404 or within the set of one or more processors 402 (e.g., within a processor's cache memory) during execution.

### Additional Considerations

Some portions of above description describe the embodiments in terms of algorithmic processes or operations. These algorithmic descriptions and representations are commonly used by those skilled in the computing arts to convey the substance of their work effectively to others skilled in the art. These operations, while described functionally, computationally, or logically, are understood to be implemented by computer programs comprising instructions for execution by a processor system (e.g., a set of one or more processors) or equivalent electrical circuits, microcode, or the like. Furthermore, it has also proven convenient at times, to refer to these arrangements of functional operations as modules, without loss of generality. The described operations and their associated modules may be embodied in software, firmware, hardware, or any combinations thereof.

Note that the components illustrated and described can include any electronics and/or computer instructions that may be embodied in digital or analog circuits. This may be implemented using any one or more of application specific integrated circuits (ASICs), field-programmable gate arrays (FPGAs), and general-purpose computing circuits, along with corresponding memories and computer program instructions for carrying out the described operations (e.g., control module 109 may be implemented using an ASIC or FPGA). The specifics of these components are not shown for clarity and compactness of description.

The term “module” is not meant to be limited to a specific physical form. Depending on the specific application, modules (e.g., 109) can be implemented as hardware, firmware, software, or any combination thereof. Furthermore, different modules can share common components or even be implemented by the same components. There may or may not be a clear boundary between different modules, even if drawn as separate elements in the figures.

Depending on the form of the components (e.g., 103, 105, 107, 109, 115), the “coupling” between them may take different forms. Dedicated circuitry can be coupled to each other by hardwiring or by accessing a common register or memory location, for example. Software “coupling” can occur by any number of ways to pass information between software components (or between software and hardware, if that is the case). The term “coupling” is meant to include all of these and is not meant to be limited to a hardwired permanent connection between two components. In addition, there may be intervening components. For example, when two components are described as being coupled to each other, this does not imply that the elements are directly coupled to each other nor does it preclude the use of other components between the two.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate +/−10% unless another meaning is apparent from the context. From example, “approximately ten” should be understood to mean “in a range from nine to eleven.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs for a system and a process for providing partial synchronization of database tables. Thus, while particular embodiments and applications have been illustrated and described, it is to be understood that the described subject matter is not limited to the precise construction and components disclosed. Various other modifications, changes and variations which will be apparent to those skilled in the art may be made in the arrangement, operation and details of the method and apparatus disclosed herein without departing from the spirit and scope as defined in the appended claims. The scope of protection should be limited only by the following claims.

What is claimed is:

1. A system comprising:
a light source with adjustable emission and positioned to illuminate an eye of a user with light;
a light detector positioned to generate data corresponding to light from the eye of the user;
a shutter with adjustable transmission and positioned between the eye of the user and an external environment; and
a control module configured to control the light source and the shutter to synchronize repeated variations of transmission of the shutter with repeated variations of emission from the light source.

2. The system of claim 1, wherein the shutter does not block propagation of the light from the light source to the eye of the user to the light detector.

3. The system of claim 1, wherein to synchronize repeated variations of the shutter with repeated variations of the light source, the control module is further configured to:
reduce light emission from the light source prior to or concurrently with increasing the transmission of the shutter; and
reduce transmission of the shutter prior to or concurrently with increasing emission of the light source.

4. The system of claim 1, further comprising:
a display positioned to display images to the eye of the user, the display having a display state when the display emits light and a dark state when the display emits a reduced amount or no light.

5. The system of claim 4, wherein the control module is further configured to synchronize repeated variations of the shutter with repeated variations of the light source and with repeated variations of the display.

6. The system of claim 4, wherein:
when the shutter is in a transmissive state:
the light source emits a reduced amount of light or no light, and
the display is in the dark state; and
when the light source emits light:
the display is in the display state, and
the shutter is in a blocking state that blocks stray or scattered light produced by the light source and the display from leaking in the external environment.

7. The system of claim 4, wherein:
when the shutter is in a transmissive state:
the light source emits a reduced amount of light or no light, and
the display is in the dark state;
when the light source emits light:
the display is in the dark state, and
the shutter is in a blocking state and blocks stray or scattered light produced by the light source from leaking in the external environment; and
when the display is in the display state:
the light source emits a reduced amount of light or no light, and
the shutter is in the blocking state and blocks stray or scattered light produced by the display from leaking in the external environment.

8. The system of claim 4, wherein:
when the shutter is in a blocking state:
at a first time period, the light source emits light and the display is in the dark state; and
at a second time period subsequent to or prior to the first time period, the light source emits a reduced amount of light or no light and the display is in the display state.

9. The system of claim 4, wherein:
at a first time period: the shutter is in a blocking state, the light source emits light, and the display is in the dark state;
at a second time period subsequent to the first time period: the shutter is in a transmissive state, the light source emits a reduced amount of light or no light, and the display is in the dark state; and
at a third time period subsequent to the first time period and the second time period: the shutter is in a blocking state, the light source emits a reduced amount of light or no light, and the display is in the display state.

10. The system of claim 4, further comprising a filter along an optical path for light reflected from the eye of the user to the light detector, the filter configured to block stray or scattered light produced by the display from propagating along the optical path toward the light detector.

11. The system of claim 4, further comprising an optical system that provides:
a first optical path for light emitted from the light source to the eye of the user, and
a second optical path for light emitted from the display to the eye of the user,
wherein the first optical path and the second optical path are the same.

12. The system of claim 4, further comprising an optical system that provides:
a first optical path for light emitted from the light source to the eye of the user, and
a second optical path for light emitted from the display to the eye of the user,
wherein the first optical path and the second optical path are different optical paths.

13. The system of claim 1, wherein the control module is further configured to synchronize the light detector generating data of the eye with repeated variations of emission from the light source.

14. The system of claim 1, further comprising a filter positioned to block stray or scattered light produced by the light source from leaking in the external environment.

15. The system of claim 1, wherein the control module is further configured to determine a gaze of the eye of the user based on data generated by the light detector.

16. The system of claim 1, wherein the light source only emits light of infrared wavelengths.

17. The system of claim 1, wherein the system is part of a head-mounted display (HMD).

18. The system of claim 1, further comprising:
an optical system that provides:
a first optical path for light from the external environment to the eye of the user,
a second optical path for light emitted from the light source to the eye of the user, and
a third optical path for light reflected from the eye of the user to the light detector.

19. A method comprising:
emitting light by a light source positioned to illuminate an eye of a user;
while the light source is emitting light:
generating data of the eye of the user by a light detector; and
blocking stray light by a shutter positioned between the eye of the user and an external environment;
reducing or ceasing to emit light by the light source; and
while the light source is emitting a reduced amount of light or no light, transitioning the shutter to a transmissive state that allows light from the external environment to propagate toward the eye of the user.

20. A non-transitory computer readable storage medium comprising instructions that, when executed by a computing system, cause the computing system to perform operations comprising:
emitting light by a light source of the computing system and positioned to illuminate an eye of a user;
while the light source is emitting light:
generating data of the eye of the user by a light detector of the computing system; and
blocking stray light by a shutter of the computing system and positioned between the eye of the user and an external environment;
reducing or ceasing to emit light by the light source; and
while the light source is emitting a reduced amount of light or no light, transitioning the shutter to a transmissive state that allows light from the external environment to propagate toward the eye of the user.
